(19) United States
(12) Patent Application Publication
(10) Pub. No.: US 2016/0306193 A1
(43) Pub. Date: Oct. 20, 2016

(54) PROTECTIVE PRESBYOPIA LENS

(71) Applicant: CHIN-JEN CHEN, TAINAN CITY (TW)

(72) Inventor: CHIN-JEN CHEN, TAINAN CITY (TW)

(21) Appl. No.: 15/194,873

(22) Filed: Jun. 28, 2016

Related U.S. Application Data
(63) Continuation-in-part of application No. 14/707,291, filed on May 8, 2015.
Application No. 14/707,291 is a continuation-in-part of application No. 13/866,230, filed on Apr. 19, 2013, now abandoned.

(30) Foreign Application Priority Data
May 21, 2012 (TW) 101209554

(51) Int. Cl. G02C 7/06; A61F 9/02

(52) U.S. Cl. CPC G02C 7/06; A61F 9/02; A61F 2009/021

(57) ABSTRACT

A protective presbyopia lens is revealed. The protection lens includes a plano lens area and a presbyopia lens area, both being produced by injection molding. The plano lens area and the presbyopia lens area are connected directly without any medium therebetween. The outer surfaces of the plano lens area and the presbyopia lens area are spheres with the same radius of curvature while the inner surfaces of the plano lens area and the presbyopia lens area are spheres with different radius of curvature. The presbyopia lens area includes upper and lower edges, left, and right edges. Both the vertical distance between the upper and lower edges and the horizontal distance between the left and the right edges are ranging from 3 cm to 5 cm. The presbyopia lens area is corresponding to and covering the user's eye while being worn. Thereby people with presbyopia can see clearly during work.

### CROSS REFERENCE TO RELATED APPLICATION

This application is a continuation-in-part of application Ser. No. 14/707,291, filed on May 8, 2015, currently pending, which is a continuation-in-part of application Ser. No. 13/866,230, filed on Apr. 19, 2013, now abandoned, which claims the benefits of the Taiwan Patent Application Serial Number 101209554, filed on May 21, 2012, the subject matter of which is incorporated herein by reference.

### BACKGROUND OF THE INVENTION

1. Fields of the invention

The present invention relates to a protective presbyopia lens, especially to a protective presbyopia lens used for eyeglasses that people with presbyopia can wear during their work.

2. Descriptions of Related Art

People have presbyopia due to aging of eyes. Thus they need to wear a corrective eyewear 1 and a protective eyewear 2 at the same time while working. As shown in FIG. 1, the protective eyewear 2 completely covers the corrective eyewear 1 for presbyopia. However, in such use mode, the user needs to wear two pairs of eyewear-the corrective eyewear 1 for presbyopia and the protective eyewear 2 at the same time. This is quite uncomfortable. While not in use, the protective eyewear 2 is taken off for storage. Thus such design is inconvenient and trouble in use and storage. Moreover, the user needs to buy both the corrective eyewear 1 for presbyopia and the protective eyewear 2 with plano lenses. Thus the cost spent on the eyewear is high.

Refer to FIG. 2, in order to solve the above problem, an eyeglass frame 5 assembled with an optical lens frame 3 and a protective lens frame 4 simultaneously has been invented and provided. A first connection slot 51 and a second connection slot 52 are mounted in the eyeglass frame 5, parallel to each other. The first connection slot 51 is used to mount the optical lens frame 3 while the second connection slot 52 is assembled with the protective lens frame 4. Thus the user can directly set the protective lens frame 4 in the eyeglass frame 5 assembled with optical lenses. He or she doesn't need to purchase another protective eyewear. A burden on the cost of the eyewear can be decreased. However, the design still has shortcomings of easy loss of components while being disassembled for storage.

Refer to U.S. Pat. No. 1,865,714, a hole is pierced in a large blank of Then a corrective lens is mounted into the hole in the blank by fusing flux. However, the processing and molding processes are complicated. The adhesion precision increases difficulty in the processing and the yield rate is reduced. There is a difference in the image due to the fusing flux. Moreover, the connection between the corrective lens and the hole is easy to be affected by thermal expansion and contraction.

Refer to U.S. Pat. No. 1,835,483, a large countersink is formed in the convex side of a blank. Then a button or disk, having a higher refractive index than the blank is fused in the countersink. Yet the melting of the corrective lens results in complicated processing procedures. The precision of the melting makes the processing and the connection more difficult. The yield rate is low. There is a difference in the image at the connection area due to different refractive index. The connection between the disk and the blank is affected by thermal expansion and contraction.

Refer to U.S. Pat. No. 6,196,678, the corrective lens and the shield of the protective glasses are not on the same plane. The corrective lenses are incorporated in the lower portion of the shield. The corrective lenses are projecting from the shield. Thus users will see an overlapped image or a difference in the image while wearing the protective glasses and this affects user's operation.

Refer to U.S. Pat. No. 6,170,952, the reading lenses are secured to the surface of a plano lens by an adhesive. The light passes the plano lens first and then the reading lenses to form an image on user's eyes. Yet different medium have different refractive index. The adhesive used affects the entrance direction of the light and causes the problem, even the reading lenses and the plano lens made from the same material.

Refer to US Pat. Pub. No. 2011/0043750 A1, a seamless bifocal lens includes a plain glass portion and a protruding region integrated with the plain glass portion. The protruding region has a central part, a border connected to the plain glass portion and surrounding the central part, and a protruding surface that protrudes gradually from the border to the central part. A thickness of the protruding surface increases from the border to the central part. Thus the appearance of the lens is seamless and the wear's vision will not be affected. However, the protruding region is located beside the user's nose and close to the left/or right side of a central horizontal line of the lens. Thus users can only see close objects clearly by a downward line of sight.

In addition, presbyopia can be corrected by using a convex lens. The protective lens of goggles should cover an eye socket of the user to meet requirements for goggles. Thus the length and the width of the protective lens should be no less than a certain size. While producing the convex lens, the protective presbyopia lens is quite thick, as shown in FIG. 3. Besides the increasing cost per cubic foot, the lens is easy to get shrunk or deformed due to the difference in thickness. Moreover, the wearer feels uncomfortable due to the consequent increased weight. Refer to FIG. 3, the thickness of a 200-degree presbyopia lens is disclosed. The thickness of the presbyopia lens is increased along with the increasing degrees. The thick presbyopia lens has shortcomings in manufacturing and wearing. Thus such lens is not well-accepted on the consumer market.

### SUMMARY OF THE INVENTION

Therefore it is a primary object of the present invention to provide a protective presbyopia lens that users can wear on occasions they need to protect their eyes. The lens has an integrated seamless appearance and the presbyopia lens area thereof is aligned with and covering the users' eye. People wear conventional presbyopia lens can only focus on close objects clearly by a downward line of sight through the presbyopia lens area. Moreover, the presbyopia lens area will not become too thick. Thus not only the lens is easy to manufacture, both the yield rate and the wearing comfort are ensured.

In order to achieve the above object, a protective presbyopia lens according to the present invention is provided.

The protection lens for people with presbyopia according to the present invention includes a plano lens area and a presbyopia lens area. Both the plano lens area and the presbyopia lens area are produced by injection molding which is performed by injection molding of an injection molding machine with a single material outlet. There is no overlap area between the plano lens area and the presbyopia lens area. The plano lens area and the presbyopia lens area are connected directly without any medium therebetween. The outer surface of the plano lens area and the outer surface of the presbyopia lens area are spheres with the same radius of curvature while the inner surface of the plano lens area and the inner surface of the presbyopia lens area are spheres with different radius of curvature. The presbyopia lens area consists of an upper edge, a lower edge, a left edge, and a right edge. Two ends of the upper edge are connected to an upper end of the left edge and an upper end of the right edge respectively while two ends of the lower edge are connected to a lower end of the left edge and a lower end of the right edge respectively. The upper edge, the lower edge, the left edge, and the right edge form a boundary of the presbyopia lens area. The vertical distance between the upper edge and the lower edge is ranging from 3 centimeters (cm) to 5 cm. So is the horizontal distance between the left edge and the right edge. When the user wears the protective glasses, the presbyopia lens area is aligned with, corresponding to and covering the user's eye.

The protective presbyopia lens according to the present invention has the following advantages:

1. The protective lens of goggles should have a certain length and width for completely covering an eye socket of the user to meet safety requirements for goggles. Yet the presbyopia lens area of the protective presbyopia lens is defined in the plano lens area of the present invention and is corresponding and covering the user's eye. Thus the convex presbyopia lens area is only a part of the protective presbyopia lens and the rest area is the plano lens area. Therefore the thickness of the convex presbyopia lens area is significantly reduced compared with the protective presbyopia lens shown in FIG. 3. Not only the cost per cubic foot can be reduced, the shrinkage and deformation generated during manufacturing process can be avoided. The light weight provides a more comfortable wearing experience.

2. The presbyopia lens area of the protective presbyopia lens is just aligned with eyes on user's face and covering user's eye. The protective presbyopia lens, of the present invention allows users to see close objects clearly without the limit of the fixed line of sight passed through the presbyopia lens area.

3. Both the plano lens area and the presbyopia lens area of the protective presbyopia lens according to the present invention are formed by an injection molding machine with a single material outlet that performs a single time injection molding. Thus there is no overlap and no medium between the plano lens area and the presbyopia lens area. The connection between the plano lens area and the presbyopia lens area will not be affected by the temperature change.

4. Both the plano lens area and the presbyopia lens area of the protective presbyopia lens according to the present invention are formed by a single time of injection molding. The injection molding is performed by an injection molding machine with a single material outlet. There is no medium therebetween. Thus the connection between the plano lens area and the presbyopia lens area are secured to each other firmly, without being affected by thermal expansion and contraction.

5. The protective presbyopia lens of the present invention provides functions of correction of presbyopia and protection. Thus the present invention has economic benefits.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a schematic drawing showing a user wearing a conventional corrective eyewear and a conventional protective eyewear at the same time;

FIG. 2 is an explosive view of a conventional eyewear frame having an optical lens frame and a protective lens frame;

FIG. 3 is a schematic drawing showing a protective presbyopia lens in a convex shape according to the present invention;

FIG. 4 is a perspective view of an embodiment of a protective presbyopia lens according to the present invention;

FIG. 5 is a cross sectional view of an embodiment of a protective presbyopia lens according to the present invention;

FIG. 6 is an embodiment of a protective presbyopia lens in use according to the present invention;

FIG. 7 is a schematic drawing application of the embodiment in FIG. 6 according to the present invention;

FIG. 8 is an explosive view of an embodiment of a protective presbyopia lens in use according to the present invention;

FIG. 9 is an assembly view of an embodiment of a protective presbyopia lens in use according to the present invention;

FIG. 10 is another assembly view of an embodiment of a protective presbyopia lens in use according to the present invention.

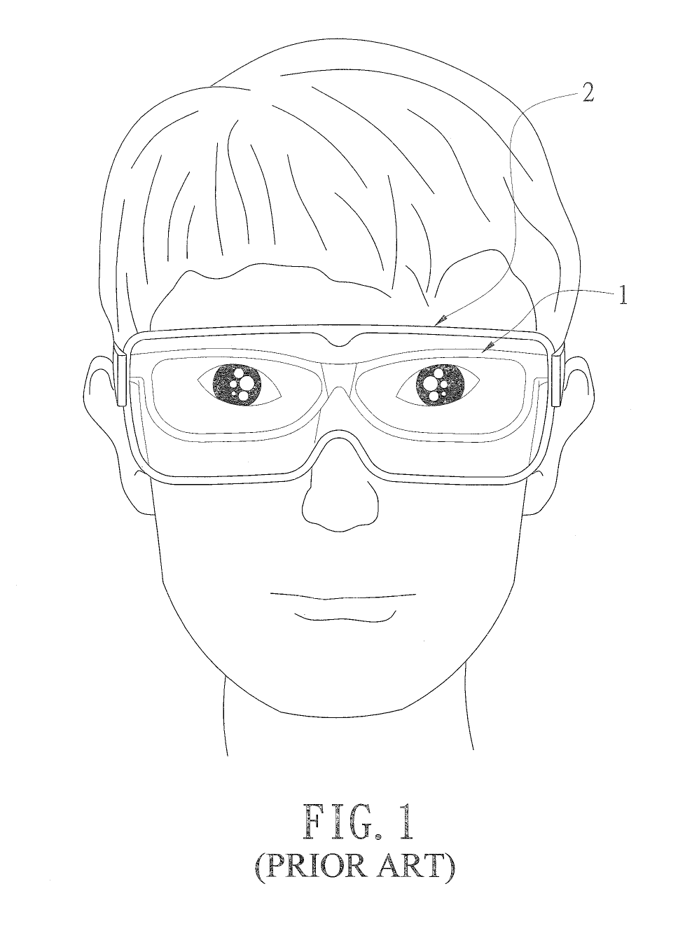

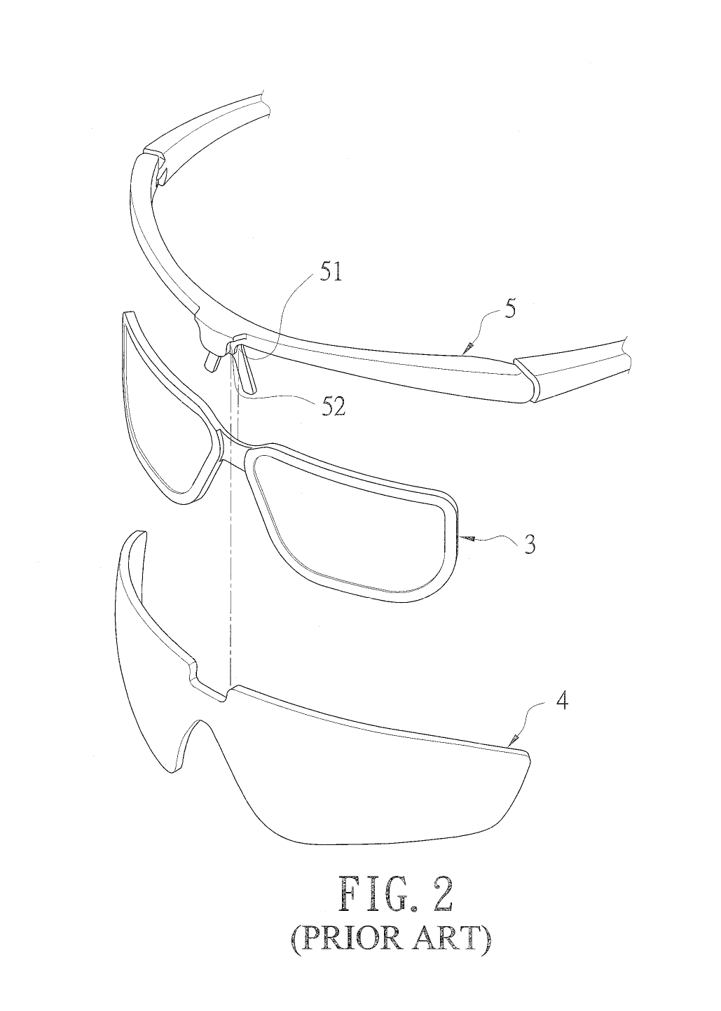

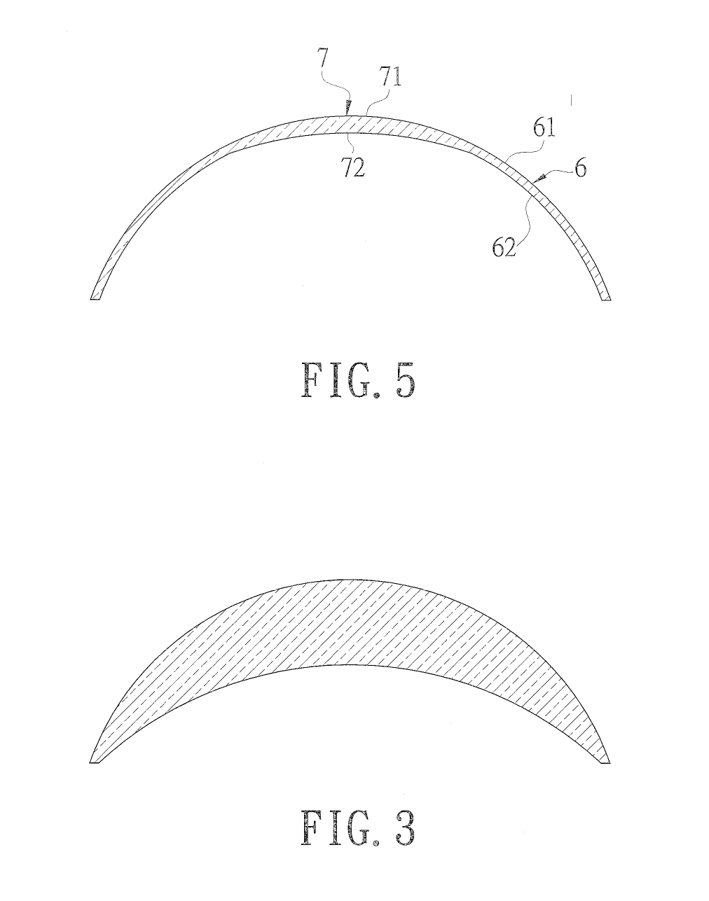

### DETAILED DESCRIPTION OF THE PREFERRED EMBODIMENT

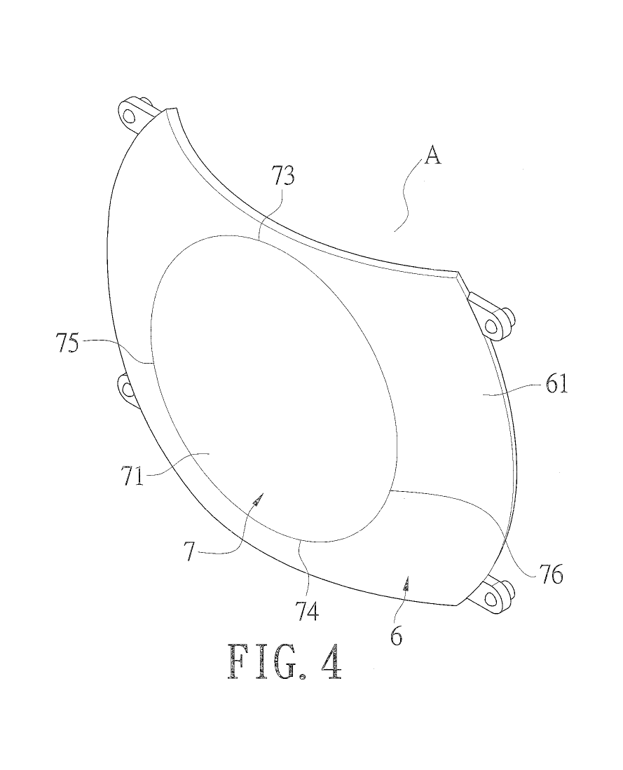

Refer to FIG. 4, a perspective view of an embodiment of a protective presbyopia lens A is revealed. The protective presbyopia lens A includes a plano lens area 6 and a presbyopia lens area 7. Both the plano lens area 6 and the presbyopia lens area 7 are produced by injection molding, especially a single time of injection molding performed by an injection molding machine with a single material outlet. The presbyopia lens area 7 is defined to be located in and connected to the plano lens area 6. The plano lens area 6 and the presbyopia lens area 7 are connected directly without any medium therebetween. At the same time, there is no overlap between the plano lens area 6 and the presbyopia lens area 7.

Both the plano lens area 6 and the presbyopia lens area 7 includes an outer surface 61/71 and an inner surface 62/72. Refer to FIG. 4 and FIG. 5, the outer surface 61/71 is the surface facing the front side of the wearer while the inner surface 62/72 is the surface facing the wearer's eyes. The outer surface 61 of the plano lens area 6 and the outer surface 71 of the presbyopia lens area 7 are connected to each other and are spheres with the same radius of curvature. The inner surface 62 of the plano lens area 6 and the inner surface 72 of the presbyopia lens area 7 are connected to each other and are spheres with different radius of curvature. The presbyopia lens area 7 includes an upper edge 73, a lower edge 74, a left edge 75, and a right edge 76. Two ends of the upper edge 73 are connected to an upper end of the left edge 75 and an upper end of the right edge 76 respectively while two ends of the lower edge 74 are connected to a lower end of the left edge 75 and a lower end of the right edge 76 respectively. The upper edge 73, the lower edge 74, the left edge 75, and the right edge 76 form a boundary of the presbyopia lens area 7. The vertical distance between the upper edge 73 and the lower edge 74 is ranging from 3 centimeters (cm) to 5 cm. The horizontal distance between the left edge 75 and the right edge 76 is ranging from 3 cm to 5 cm. The presbyopia lens area 7 is corresponding to and covering the user's eye when the user wears the protective presbyopia lens.

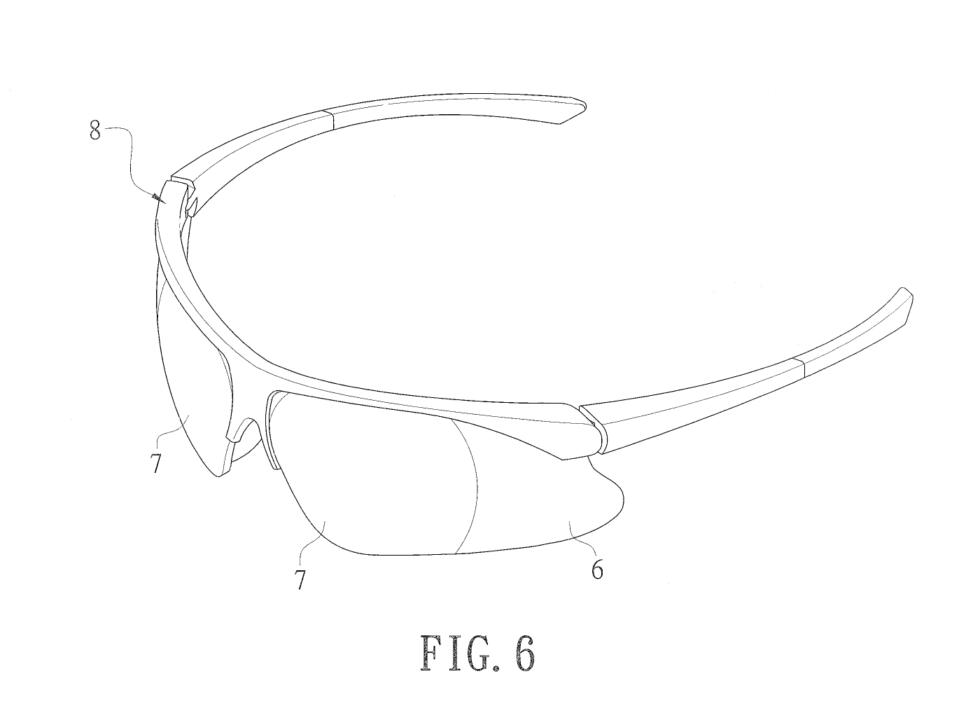

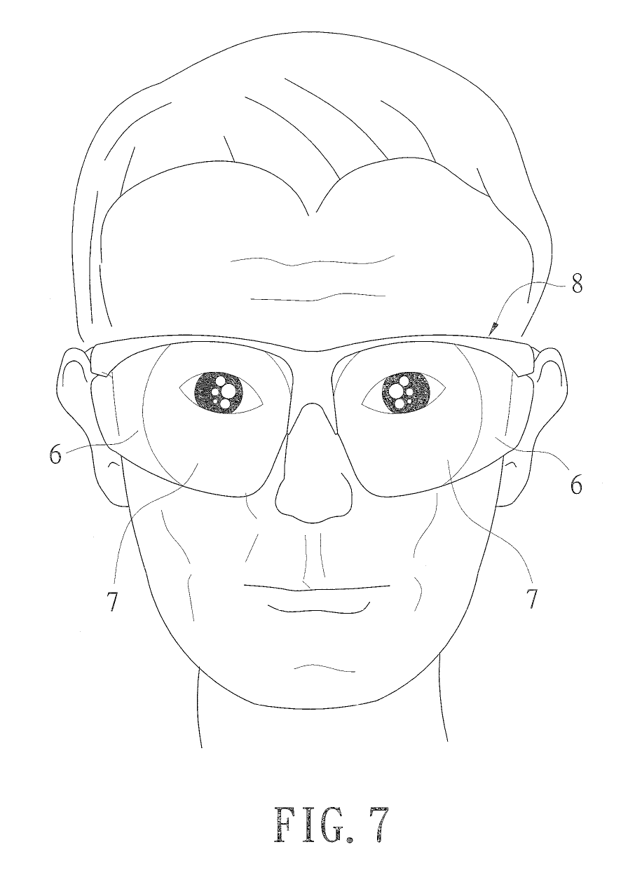

Refer to FIG. 6 and FIG. 7, the protective presbyopia lens A is cut according to the shape of a frame 8 and assembled on the frame 8. When the user wear the protective presbyopia lens A, the presbyopia lens area 7 is corresponding to and covering the user's eye, as shown in FIG. 7. Thus the user can see the near object clearly and his/her eyes are protected.

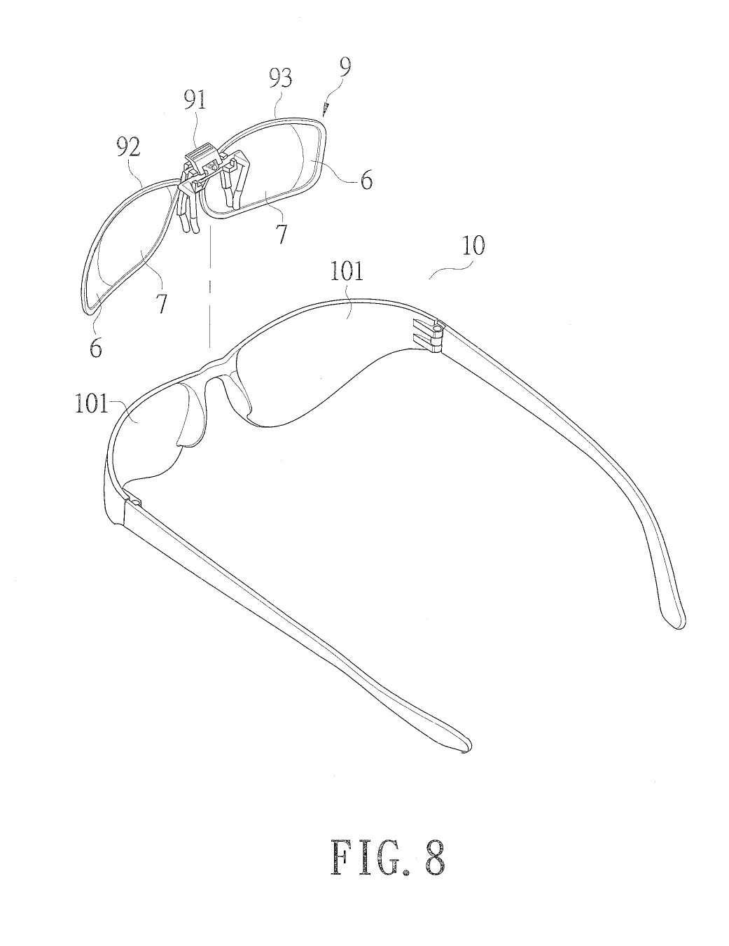

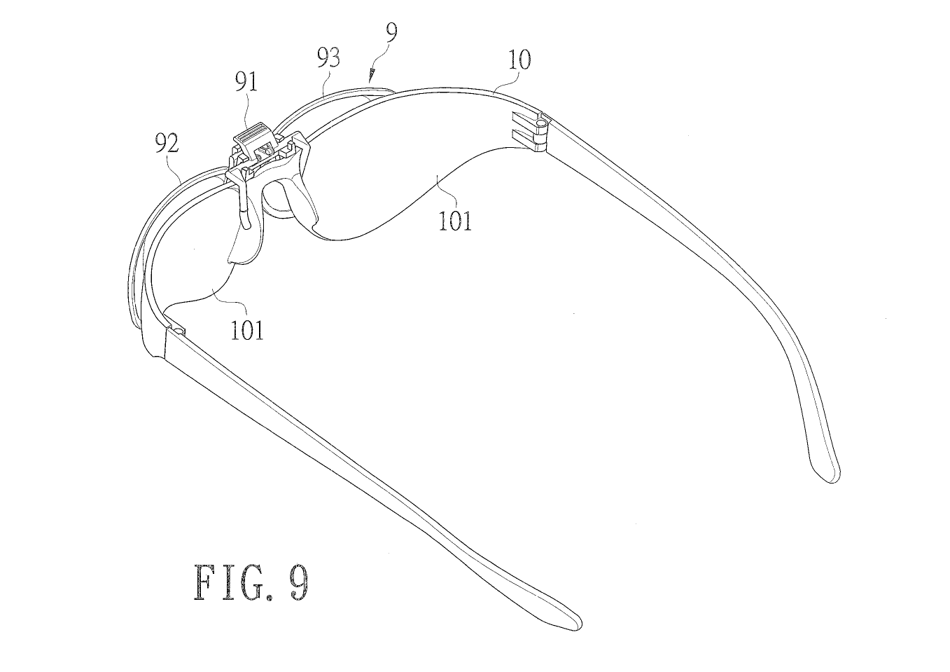

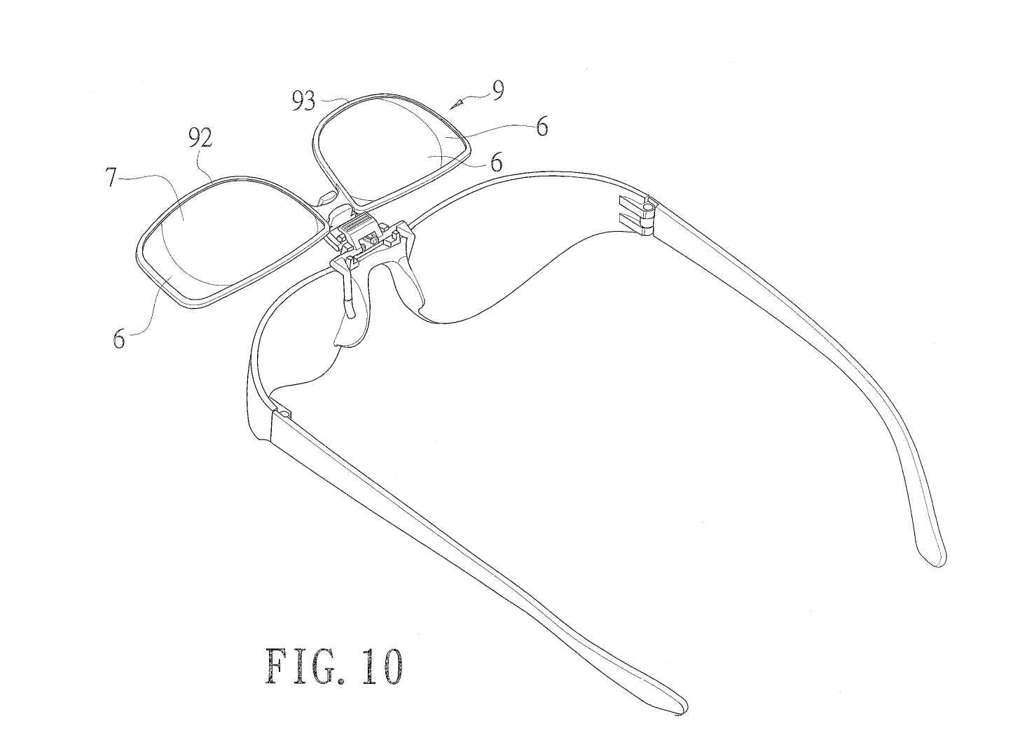

Refer to FIG. 8, FIG. 9 and FIG. 10, a fixture 9 is pivotally connected to clip-on reading glasses 9. Thus the clip-on reading glasses 9 is fixed and clipped on safety glasses 10 available on the market by a clip 91. The lenses 101 of the safety glasses 10 are plano lenses. The clip-on reading glasses 9 include two lens parts 92, 93 formed by direct injection. Each lens part 92/93 includes a plano lens area 6 and a presbyopia lens area 7. Thereby people with presbyopia can see close objects clearly by the clip-on reading glasses 9 with two injection-molded lens parts 92, 93 being attached to the safety glasses 10 with plano lenses available on the market, without lifting the clip-on reading glasses 9.

In the presbyopia lens area 7, the vertical distance between the upper edge 73 and the lower edge 74 and the horizontal distance between the left edge 75 and the right edge 76 are both ranging from 3 cm to 5 cm so that the presbyopia lens area 7 is just aligned with user's eye while being worn by the user. The presbyopia lens area 7 is still covering the user's eye even user's pupil is moved along with the sight. Thus the user can see close objects clearly, without affecting the work.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A protective presbyopia lens comprising a plano lens area and a presbyopia lens area;
wherein both the plano lens area and the presbyopia lens area are produced by an injection molding machine having a single material outlet that performs a single time of injection molding; the presbyopia lens area is defined to be located in and connected to the plano lens area; there is no overlap between the plano lens area and the presbyopia lens area while the plano lens area and the presbyopia lens area are connected directly without any medium therebetween; wherein an outer surface of the plano lens area and an outer surface of the presbyopia lens area are spheres with the same radius of curvature while an inner surface of the plano lens area and an inner surface of the presbyopia lens area are spheres with different radius of curvature; the presbyopia lens area includes an upper edge, a lower edge, a left edge, and a right edge; two ends of the upper edge are connected to an upper end of the left edge and an upper end of the right edge respectively while two ends of the lower edge are connected to a lower end of the left edge and a lower end of the right edge respectively;
the upper edge, the lower edge, the left edge, and the right edge form a boundary of the presbyopia lens area; a vertical distance between the upper edge and the lower edge is ranging from 3 centimeters (cm) to 5 cm;
a horizontal distance between the left edge and the right edge is ranging from 3 cm to 5 cm; the presbyopia lens area is corresponding to and covering the user's eye when the user wears the protective presbyopia lens.
